(19) United States
(12) Patent Application Publication
(10) Pub. No.: US 2008/0293942 A1
(43) Pub. Date: Nov. 27, 2008

(54) Methods of Preparing 2-Imidazol-1-Yl-4-Methyl-6-Pyrrolidin-2-Yl-Pyrimidine and 4-(1-Alkylpyrrolidin-2-Yl)-2-(1H-Imidazol-1-Yl)-6-Methylpyrimidine Derivatives

(76) Inventor: Russell D. Cousins, San Diego, CA (US)

Correspondence Address: GLOBAL PATENT GROUP - KAL;ATTN: MS LAVERN HALL, 10411 Clayton Road, Suite 304, St. Louis, MO, 63131, US

(21) Appl. No.: 12/095,293

(22) PCT Filed: Nov. 27, 2006
(86) PCT No.: PCT/US06/61262

§ 371 Date: May 28, 2008

Related U.S. Application Data
(60) Provisional application No. 60/740,531, filed on Nov. 28, 2005.

(51) Int. Cl. C07D 403/14; C07D 403/04; C07D 207/16

(52) U.S. Cl. 544/331; 544/330; 548/536

(57) ABSTRACT

The present invention is directed to a novel, high yield method for preparing 2-imidazol-1-yl-4-methyl-6-pyrrolidin-2-yl-pyrimidine, particularly to a method of preparing 4-(1-alkylpyrrolidin-2-yl)-2-(1H-imidazol-1-yl)-6-methylpyrimidine, more particularly, 2-(2-(2-(1H-imidazol-1-yl)-6-methylpyrimidin-4-yl)pyrrolidrn-1-yl)-N-(benzo[d][1,3]dioxol-5-yh-nethyl)-N-methylethanamine. These compounds and pharmaceutical compositions thereof are inhibitors of nitric oxide synthase, are selective for inducible nitric oxide synthase, and are useful in treating diseases and disorders including inflammation and pain.

This application claims the benefit of priority of U.S. provisional application No. 60/740, 531, filed Nov. 28, 2005, the disclosure of which is hereby incorporated by reference as if written herein in its entirety.

### FIELD OF THE INVENTION

This invention relates to a process for preparing 2-imidazol-1-yl-4-methyl-6-pyrrolidin-2-yl-pyrimidine, particularly to a method of preparing 4-(1-alkylpyrrolidin-2-yl)-2-(1H-imidazol-1-yl)-6-methylpyrimidine, more particularly, 2-(2-(2-(1H-imidazol-1-yl)-6-methylpyrimidin-4-yl)pyrrolidin-1-yl)-N-(benzo[d][1,3]dioxol-5-ylmethyl)-N-methylethanamine, that afford a high yield of pure product.

### BACKGROUND OF THE INVENTION

It has been found that 4-(1-ethylpyrrolidin-2-yl)-2-(1H-imidazol-1-yl)-6-methylpyrimidine derivatives may be obtained in a method wherein N-Z-D,L-proline is converted into a compound having a 1,3-diketone group in the extended side-chain, which is then cyclized and dehydrated using guanidine, and then converted to an imidazole through a cyclization and dehydration procedure, which is N-deprotected and N-alkylated.

### SUMMARY OF THE INVENTION

This invention is directed to a novel, high yield process for preparing substituted 2-imidazol-1-yl-4-methyl-6-pyrrolidin-2-yl-pyrimidine derivatives, particularly to a method of preparing 4-(1-alkylpyrrolidin-2-yl)-2-(1H-imidazol-1-yl)-6-methylpyrimidine, more particularly 2-(2-(2-(1H-imidazol-1-yl)-6-methylpyrimidin-4-yl)pyrrolidin-1-yl)-N-(benzo[d][1,3]dioxol-5-ylmethyl)-N-methylethanamine. According to the process disclosed herein, N-Z-D,L-proline is converted into a compound having a 1,3-diketone group in the extended side-chain, which is then cyclized and dehydrated using guanidine, then converted to an imidazole through a cyclization and dehydration procedure, which is N-deprotected and N-alkylated.

### DETAILED DESCRIPTION OF THE INVENTION

This invention relates to a novel method for preparing in high yield 4-(1-alkylpyrrolidin-2-yl)-2-(1H-imidazol-1-yl)-6-methylpyrimidine derivatives of structural formula (I),

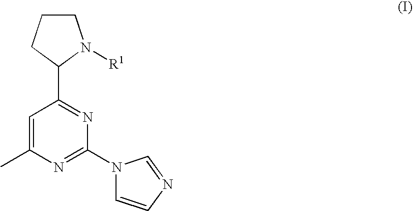

wherein:

R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboarylalkoxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted as defined herein.

Said novel method comprises:
a) treating N—R¹-D,L-proline, of structural formula (II),

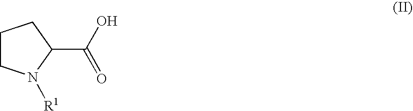

with suitable reagents, including, but not limited to, an excess oxalyl chloride and a catalytic amount of DMF in a suitable solvent; an excess of thionyl chloride and a catalytic amount of DMF in a suitable solvent; stoichiometric amount of oxalyl chloride and a catalytic amount of DMF in a suitable solvent; stoichiometric amount of thionyl chloride and a catalytic amount of DMF in a suitable solvent; with or without isolating the novel reaction product of structural formula (III),

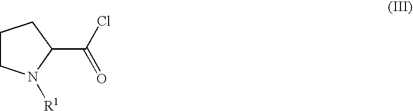

then
b) reacting the reaction product of step (a), the compound of structural formula (III), with an excess of the salt of a monoanion of alkylacetoacetate, as defined below, in a suitable solvent, at a temperature of from −20° C. to reflux temperature for from 5 minutes to 48 hours, with or without isolating the novel reaction product of structural formula (IV),

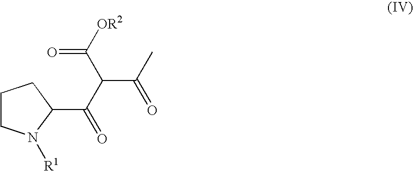

then
c) reacting the reaction product of step (b), the compound of structural formula (IV), with an excess of protic acid, as defined below, in a suitable solvent, at temperature of from −20° C. to reflux for from 30 minutes to 48 hours; with or without isolating the reaction product of structural formula (V),

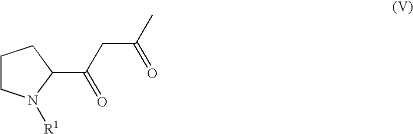

then
d) reacting the reaction product of step (c), the compound of structural formula (V), with suitable reagents, including, but not limited to, an excess of an inorganic salt of guanidine and an inorganic base in a suitable solvent; an excess of an inorganic salt of guanidine and an organic base in a suitable solvent; a stoichiometric amount of an inorganic salt of guanidine and an inorganic base in a suitable solvent; a stoichiometric amount of an inorganic salt of guanidine and an organic base in a suitable solvent; a stoichiometric amount of guanidine in a suitable solvent; with or without isolating the reaction product of structural formula (VI),

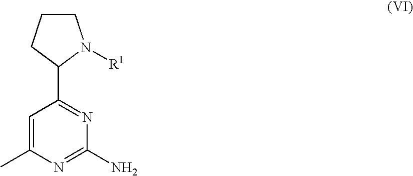

and
e) reacting the reaction product of step (d), the compound of structural formula (VI), with suitable reagents, including, but not limited to, an excess of formalin, glyoxal, ammonium chloride, and an appropriate amount of protic acid in a suitable solvent; an excess of paraformaldehyde, glyoxal, ammonium chloride, and an appropriate amount of protic acid in a suitable solvent, or stoichiometric amounts paraformaldehyde, glyoxal, ammonium chloride and a catalytic amount of phosphoric acid; and isolating the novel reaction product of structural formula (I) in high purity.

The starting material of structural formula (II) may be prepared by standard methods known to those skilled in the art, by alkylation of D,L-proline to give the N-alkyl-D,L-proline.

It will be obvious to one skilled in the art that the R¹ group in compound of structural formula (II) may be a protecting group, such as benzyloxycarbonyl, tert-butyloxycarbonyl, methoxycarbonyl, or formyl, for example. Such a starting material may be carried through steps (a) and (e) of the process described above, to give a compound similar to that of structural formula (I), wherein R¹ is a protecting group. Subsequently, the protecting group may be removed and the R¹ group added to give the desired compound of structural formula (I). Such an extension of the process is to be considered within the scope of the present invention.

One embodiment of the process for preparing structural compound (I) comprises:

reacting the compound of structural formula (IIa),

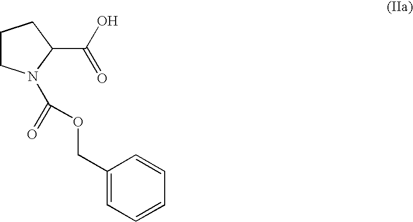

a) with an excess of oxalyl chloride and a catalytic amount of DMF in a suitable solvent at a temperature of from −20° C. to 80° C. for from 5 minutes to 48 hours, vigorously mixing of reagents, and optionally isolating the novel compound of structural formula (IIIa),

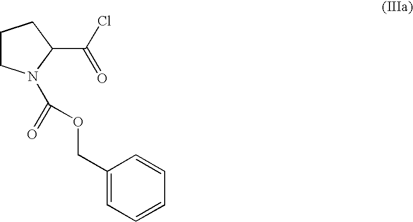

then
b) reacting the compound of structural formula (IIIa) with an excess of the inorganic salt of the monoanion of an alkyl acetoacetate in a suitable solvent at a temperature of from −20° C. to 80° C. for from 5 minutes to 48 hours, vigorously mixing of reagents, and optionally isolating the novel compound of structural formula (VII),

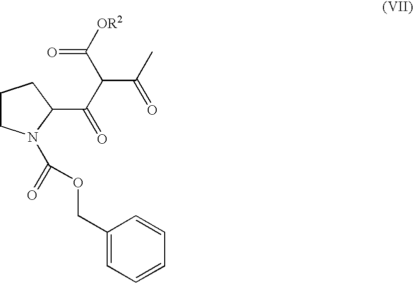

wherein R² is optionally substituted alkyl;

then
c) reacting of compound of structural formula (VII), wherein R²=tert-butoxy, with an excess of protic acid, as defined below, in a suitable solvent, at temperature of from −20° C. to reflux for from 30 minutes to 48 hours; with or without isolating the reaction product of structural formula (VIII),

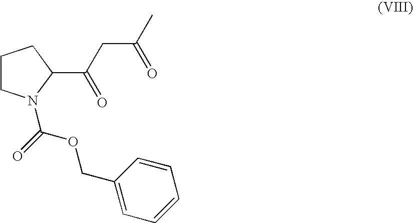

then
d) reacting of the compound of structural formula (VIII), with suitable reagents, including, but not limited to, an excess of an inorganic salt of guanidine and an inorganic base in a suitable solvent; an excess of an inorganic salt of guanidine and an organic base in a suitable solvent; a stoichiometric amount of an inorganic salt of guanidine and an inorganic base in a suitable solvent; a stoichiometric amount of an inorganic salt of guanidine and an organic base in a suitable solvent; a stoichiometric amount of guanidine in a suitable solvent; with or without isolating the reaction product of structural formula (IX),

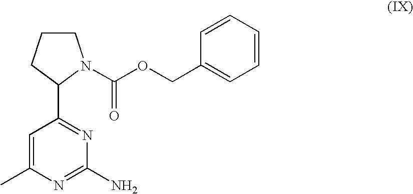

then
e) reacting the compound of structural formula (IX), with suitable reagents, including, but not limited to, an excess of formalin, glyoxal, ammonium chloride, and an appropriate amount of protic acid, as defined below, in a suitable solvent; an excess of paraformaldehyde, glyoxal, ammonium chloride, and an appropriate amount of protic acid in a suitable solvent, or stoichiometric amounts of paraformaldehyde, glyoxal, ammonium chloride and a catalytic amount of phosphoric acid; and isolating the protected intermediate product of structural formula (X),

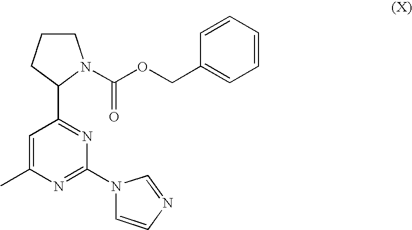

then
f) reacting the compound of structural formula (X), with appropriate hydrogenation agents in a suitable solvent; with or without isolating the reaction product of structural formula (XI),

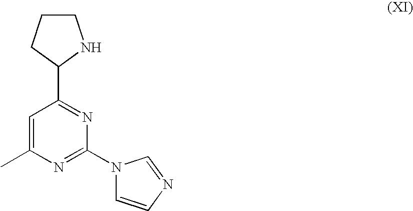

and
g) reacting of the compound of structural formula (XI), with an N-alkylating agent, as defined below, in a suitable solvent, at a temperature of from ambient to reflux for from 1 hour to 48 hours and isolating the novel reaction product of structural formula (I) in high purity.

Another embodiment is the process for preparing compound of structural formula (XII),

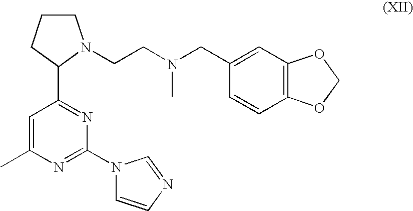

comprising:
a) reacting the compound of structural formula (IIa),

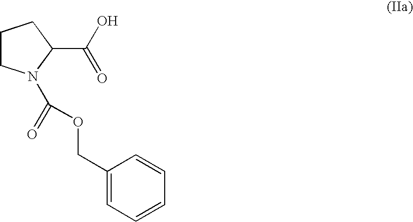

with an excess of oxalyl chloride and a catalytic amount of DMF in methylene chloride at a temperature of from 0° C. to ambient for from 5 minutes to 16 hours, vigorously mixing of reagents, and optionally isolating the novel compound of structural formula (IIIa),

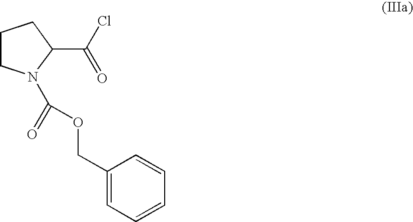

then
b) reacting the compound of structural formula (IIIa) with an excess of the magnesium salt of the monoanion of tert-butylacetoacetate in THF at a temperature of from 0° C. to ambient for from 5 minutes to 24 hours, vigorously mixing of reagents, and optionally isolating the novel compound of structural formula (VIIa),

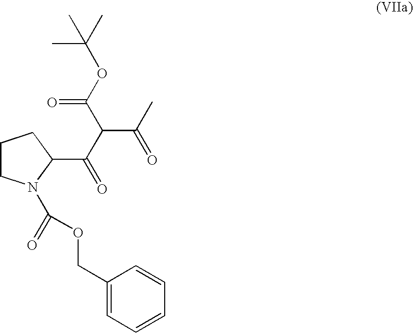

then
c) reacting of compound of structural formula (VIIa) with a stoichiometric amount of para-toluenesulfonic acid monohydrate in toluene; a catalytic amount of para-toluenesulfonic acid monohydrate in toluene; at temperature of from ambient to reflux for from 30 minutes to 20 hours; with or without isolating the reaction product of structural formula (VIII),

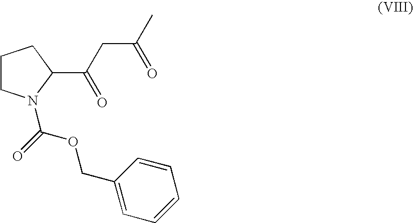

then
d) reacting of the compound of structural formula (VIII) with a stoichiometric amount of guanidine in ethanol at a temperature of from ambient to reflux for from 1 hour to 48 hours; with or without isolating the reaction product of structural formula (IX),

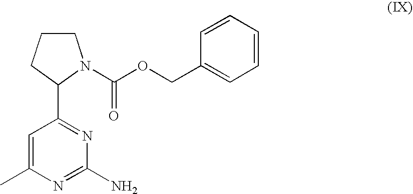

then
e) reacting the compound of structural formula (IX) with excess amounts of paraformaldehyde, glyoxal, ammonium chloride and a suitable amount of phosphoric acid in a water/dioxane solvent mixture at a temperature of from ambient to reflux for from 1 hour to 48 hours; and isolating the protected intermediate product of structural formula (X),

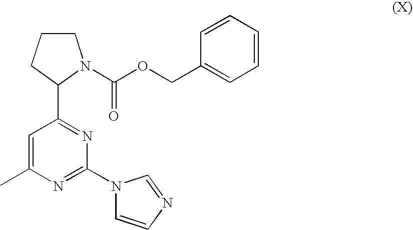

then
f) reacting the compound of structural formula (X) with a catalyst, such as Pd/C or Pt/C, and appropriate amount of hydrogen in a suitable solvent at a pressure of from 1 atm to 20 atm for from 10 minutes to 48 hours; with or without isolating the reaction product of structural formula (XI),

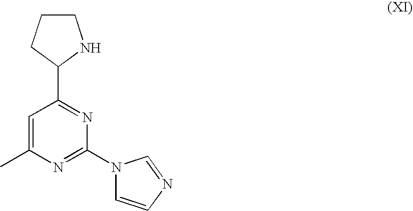

and
g) reacting of the compound of structural formula (XI), with the alkylating agent benzo[1,3]dioxol-5-ylmethyl-(2-chloro-ethyl)-methyl-amine hydrochloride salt, in the presence of a tertiary amine base and a halide salt in a suitable solvent at a temperature of from ambient to reflux for from 1 hour to 48 hours and isolating the novel reaction product of structural formula (XII) in high purity.

One embodiment of the present invention is the method for preparing the compound of structural formula (III):

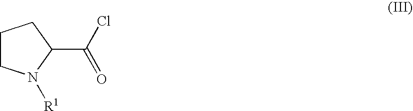

comprising the steps of:
a) treating a compound of structural formula (II),

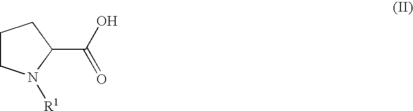

with a combination of suitable reagents, comprising a halogenating reagent, a catalytic amount of N,N-dimethylformamide, in a suitable solvent, using an appropriate reaction time over a suitable temperature range reagents; and
b) with or without isolating the novel reaction product of structural formula (III).

One embodiment of the present invention is the method for preparing the compound of structural formula (III), wherein oxalyl chloride and thionyl chloride are the halogenating agents, either of which may be present in an amount greater than or equal to a stoichiometric amount.

One embodiment of the present invention is the method for preparing the compound of structural formula (III), wherein oxalyl chloride is the halogenating agent.

One embodiment of the present invention is the method for preparing the compound of structural formula (III), wherein said oxalyl chloride is present in greater than stoichiometric amounts.

One embodiment of the present invention is the method for preparing the compound of structural formula (III), wherein said solvent is selected from the group consisting of dichloromethane, 1,2-dichloroethane, chloroform, benzene, toluene, xylene; or any of these may be combined together and utilized as co-solvents.

One embodiment of the present invention is the method for preparing the compound of structural formula (III), wherein said solvent is dichloromethane.

One embodiment of the present invention is the method for preparing the compound of structural formula (III), wherein the suitable temperature range is from about −20° C. to 40° C.

One embodiment of the present invention is the method for preparing the compound of structural formula (III), wherein said suitable temperature range is from 0° C. to ambient temperature.

One embodiment of the present invention is the method for preparing the compound of structural formula (III), wherein the appropriate reaction time range is from about 5 minutes to 24 hours.

One embodiment of the present invention is the method for preparing the compound of structural formula (III), wherein said reaction time range is from 12 to 16 hours.

Another embodiment of the present invention is the method for preparing the compound of structural formula (IV):

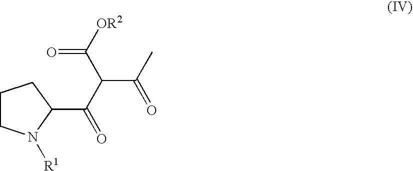

or a salt, ester, or prodrug thereof, wherein:

R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted;

R² is optionally substituted alkyl;

comprising:
a) treating the reaction product of structural formula (III),

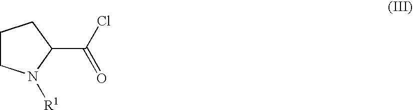

with an excess of the salt of a monoanion of alkylacetoacetate, in a suitable solvent, using an appropriate reaction time over a suitable temperature range; and
b) isolating the novel reaction product of structural formula (IV).

Another embodiment of the present invention is the method for preparing the compound of structural formula (IV), wherein said salt is selected from the group consisting of sodium, potassium, magnesium, and calcium.

Another embodiment of the present invention is the method for preparing the compound of structural formula (IV), wherein the salt is the magnesium salt.

Another embodiment of the present invention is the method for preparing the compound of structural formula (IV), wherein said solvent is selected from the group consisting of tetrahydrofuran, diethyl ether, dioxane, and dimethoxyethane; or any of these may be combined together and utilized as co-solvents.

Another embodiment of the present invention is the method for preparing the compound of structural formula (IV), wherein said solvent is tetrahydrofuran.

Another embodiment of the present invention is the method for preparing the compound of structural formula (IV), wherein the suitable temperature range is from about −70° C. to 80° C.

Another embodiment of the present invention is the method for preparing the compound of structural formula (IV), wherein said suitable temperature range is from 0° C. to ambient temperature.

Another embodiment of the present invention is the method for preparing the compound of structural formula (IV), wherein the appropriate reaction time range is from about 5 minutes to 24 hours.

Another embodiment of the present invention is the method for preparing the compound of structural formula (IV), wherein said reaction time range is from 10 to 14 hours.

A further embodiment of the present invention is the method for preparing the compound of structural formula (V):

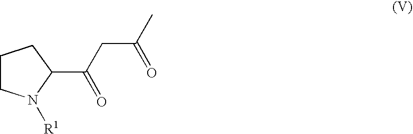

or a salt, ester, or prodrug thereof, wherein:

R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted;

comprising:
a) treating the reaction product of structural formula (IV),

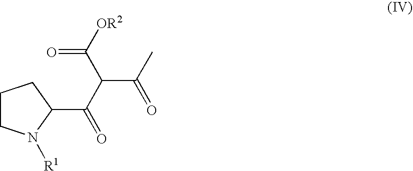

with a protic acid, in a suitable solvent, using an appropriate reaction time over a suitable temperature range; and in a suitable solvent; and
b) isolating the novel reaction product of structural formula (IV).

A further embodiment of the present invention is the method for preparing the compound of structural formula (V), wherein said protic acid is selected from the group consisting of hydrogen chloride, p-toluenesulfonic acid, benzenesulfonic acid, camphorsulfonic acid, and trifluoroacetic acid, any of which may be present in an amount ranging from catalytic to a stoichiometric amount.

A further embodiment of the present invention is the method for preparing the compound of structural formula (V), wherein the protic acid is p-toluenesulfonic acid.

A further embodiment of the present invention is the method for preparing the compound of structural formula (V), wherein p-toluenesulfonic acid is present in catalytic amounts.

A further embodiment of the present invention is the method for preparing the compound of structural formula (V), wherein said solvent is selected from the group consisting of benzene, toluene, xylene, and dichloroethane; or any of these may be combined together and utilized as co-solvents.

A further embodiment of the present invention is the method for preparing the compound of structural formula (V), wherein said solvent is toluene.

A further embodiment of the present invention is the method for preparing the compound of structural formula (V), wherein the suitable temperature range is from about −25° C. to 140° C.

A further embodiment of the present invention is the method for preparing the compound of structural formula (V), wherein said suitable temperature range is from 70° C. to 90° C.

A further embodiment of the present invention is the method for preparing the compound of structural formula (V), wherein the appropriate reaction time range is from about 5 minutes to 24 hours.

A further embodiment of the present invention is the method for preparing the compound of structural formula (V), wherein said reaction time range is from 2 to 6 hours.

Another embodiment of the present invention is the method for preparing the compound of structural formula (VI):

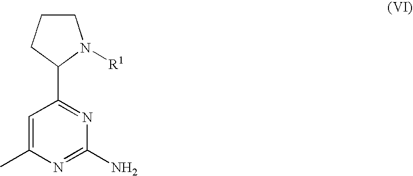

or a salt, ester, or prodrug thereof, wherein:

R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted;

comprising:
a) a treating the reaction product of structural formula (V),

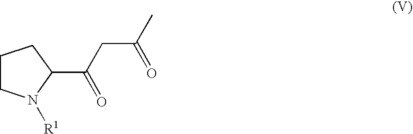

with suitable reagents including, but not limited to, an excess of an inorganic salt of guanidine, and an inorganic base, in a suitable solvent, using an appropriate reaction time over a suitable temperature range; and
b) optionally isolating the reaction product of structural formula (VI).

Another embodiment of the present invention is the method for preparing the compound of structural formula (VI), wherein said inorganic salt of guanidine is selected from the group consisting of guanidine hydrochloride, guanidine carbonate, and guanidine sulfate, any of which may be present in an amount greater than or equal to a stoichiometric amount.

Another embodiment of the present invention is the method for preparing the compound of structural formula (VI), wherein the inorganic salt is guanidine hydrochloride.

Another embodiment of the present invention is the method for preparing the compound of structural formula (VI), wherein said guanidine hydrochloride is present in greater than a stoichiometric amount.

Another embodiment of the present invention is the method for preparing the compound of structural formula (VI), wherein said solvent is selected from the group consisting of methanol, ethanol, 1-propanol, 2-propanol, 1-butanol, and tert-butanol; or any of these may be combined together and utilized as co-solvents.

Another embodiment of the present invention is the method for preparing the compound of structural formula (VI), wherein said solvent is ethanol.

Another embodiment of the present invention is the method for preparing the compound of structural formula (VI), wherein the suitable temperature range is from about 0° C. to 120° C.

Another embodiment of the present invention is the method for preparing the compound of structural formula (VI), wherein said suitable temperature range is from 70° C. to 90° C.

Another embodiment of the present invention is the method for preparing the compound of structural formula (VI), wherein the appropriate reaction time range is from about 5 minutes to 24 hours.

Another embodiment of the present invention is the method for preparing the compound of structural formula (VI), wherein said reaction time range is from 10 to 14 hours.

A further embodiment of the present invention is the method for preparing the compound of structural formula (I)

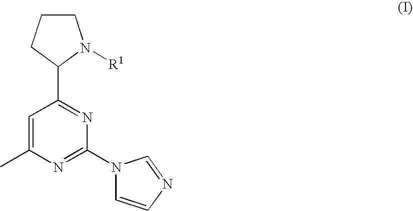

or a salt, ester, or prodrug thereof, wherein:

R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted;

comprising:
a) a treating the reaction product of structural formula (VI),

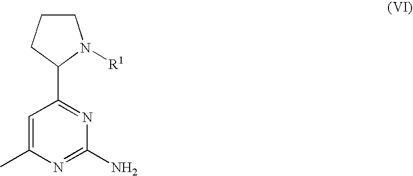

with a combination of suitable reagents, comprising a formaldehyde equivalent, a glyoxal equivalent, a ammonia equivalent, and an appropriate amount of a protic acid in a suitable protic solvent, using an appropriate reaction time over a suitable temperature range;
b) isolating the novel reaction product of structural formula (I) in high yield and purity;
c) optionally removing the R¹ group to afford a compound of structural formula (I), wherein R¹ is hydrogen; and
d) optionally alkylating the compound of structural formula (I), wherein R¹ is hydrogen, with an appropriate alkylating agent in a suitable solvent, using an appropriate reaction time over a suitable temperature range

A further embodiment of the present invention is the method for preparing the compound of structural formula (I), wherein said formaldehyde equivalent is formalin or paraformaldehyde, either of which may be present in an amount greater than or equal to a stoichiometric amount.

A further embodiment of the present invention is the method for preparing the compound of structural formula (I), wherein paraformaldehyde is utilized as a reagent.

A further embodiment of the present invention is the method for preparing the compound of structural formula (I), wherein said paraformaldehyde is present in stoichiometric amounts.

A further embodiment of the present invention is the method for preparing the compound of structural formula (I), wherein said glyoxal equivalent is anhydrous glyoxal or glyoxal hydrate, either of which may be present in an amount greater than or equal to a stoichiometric amount.

A further embodiment of the present invention is the method for preparing the compound of structural formula (I), wherein glyoxal hydrate is employed as a reagent.

A further embodiment of the present invention is the method for preparing the compound of structural formula (I), wherein said glyoxal hydrate is present in stoichiometric amounts.

A further embodiment of the present invention is the method for preparing the compound of structural formula (I), wherein said ammonia equivalent is selected from the group consisting of ammonium chloride, ammonia gas, ammonium hydroxide, ammonium acetate, ammonium sulfate, ammonium bicarbonate, and ammonium carbamate, any of which may be present in an amount greater than or equal to a stoichiometric amount.

A further embodiment of the present invention is the method for preparing the compound of structural formula (I), wherein ammonium chloride is employed as a reagent.

A further embodiment of the present invention is the method for preparing the compound of structural formula (I), wherein said ammonium chloride is present in stoichiometric amounts.

A further embodiment of the present invention is the method for preparing the compound of structural formula (I), wherein said protic acid is phosphoric acid, present in an amount ranging from catalytic to greater than or equal to a stoichiometric amount.

A further embodiment of the present invention is the method for preparing the compound of structural formula (I), wherein said phosphoric acid is present in stoichiometric amounts.

A further embodiment of the present invention is the method for preparing the compound of structural formula (I), wherein said phosphoric acid is present in catalytic amounts.

A further embodiment of the present invention is the method for preparing the compound of structural formula (I), wherein:

said solvent is selected from the group consisting of water, dioxane, ethanol, methanol, 1-propanol, 2-propanol, 1-butanol, tert-butanol, methoxyethanol, ethoxyethanol, ethylene glycol, and propylene glycol; or any of these protic solvents may be combined together and utilized as co-solvents.

A further embodiment of the present invention is the method for preparing the compound of structural formula (I), wherein dioxane and water are employed as the solvents.

A further embodiment of the present invention is the method for preparing the compound of structural formula (I), wherein the suitable temperature range is from about 0° C. to 150° C.

A further embodiment of the present invention is the method for preparing the compound of structural formula (I), wherein said suitable temperature range is from 80° C. to 110° C.

A further embodiment of the present invention is the method for preparing the compound of structural formula (I), wherein said reaction time range is from about 5 minutes to 48 hours.

A further embodiment of the present invention is the method for preparing the compound of structural formula (I), wherein said reaction time range is from 0.5 to 4 hours.

In yet further embodiments of the present invention is the method for preparing the compound of structural formula (VII):

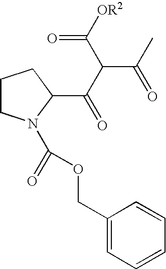

or a salt, ester, or prodrug thereof, wherein:

R² is optionally substituted alkyl;

comprising:
a) treating the reaction product of structural formula (IIIa),

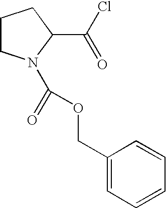

with an excess of the salt of a monoanion of alkylacetoacetate, in a suitable solvent, using an appropriate reaction time over a suitable temperature range reagents; and
b) isolating the novel reaction product of structural formula (IV).

In yet further embodiments of the present invention is the method for preparing the compound of structural formula (VII), wherein said salt is selected from the group consisting of sodium, potassium, magnesium, and calcium.

In yet further embodiments of the present invention is the method for preparing the compound of structural formula (VII), wherein the salt is the magnesium salt.

In yet further embodiments of the present invention is the method for preparing the compound of structural formula (VII), wherein said solvent is selected from the group consisting of tetrahydrofuran, diethyl ether, dioxane, and dimethoxyethane; or any of these may be combined together and utilized as co-solvents.

In yet further embodiments of the present invention is the method for preparing the compound of structural formula (VII), wherein said solvent is tetrahydrofuran.

In yet further embodiments of the present invention is the method for preparing the compound of structural formula (VII), wherein the suitable temperature range is from about −70° C. to 80° C.

In yet further embodiments of the present invention is the method for preparing the compound of structural formula (VII), wherein said suitable temperature range is from 0° C. to ambient temperature.

In yet further embodiments of the present invention is the method for preparing the compound of structural formula (VII), wherein the appropriate reaction time range is from about 5 minutes to 24 hours.

In yet further embodiments of the present invention is the method for preparing the compound of structural formula (VII), wherein said reaction time range is from 10 to 14 hours.

In other embodiments of the present invention is the method for preparing the compound of structural formula (VIII):

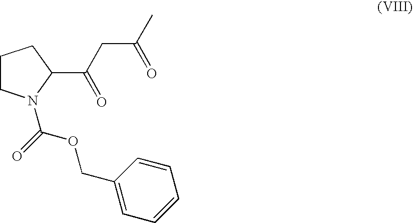

or a salt, ester, or prodrug thereof, comprising:
a) treating the reaction product of structural formula (VII),

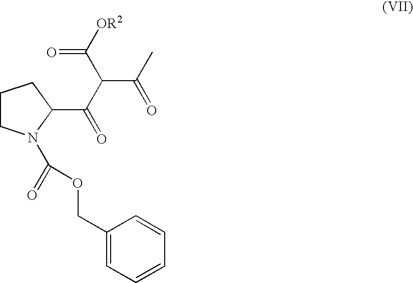

with a protic acid, in a suitable solvent, using an appropriate reaction time over a suitable temperature range; and in a suitable solvent; and
b) isolating the novel reaction product of structural formula (VIII).

In other embodiments of the present invention is the method for preparing the compound of structural formula (VIII), wherein said protic acid is selected from the group consisting of hydrogen chloride, p-toluenesulfonic acid, benzenesulfonic acid, camphorsulfonic acid, and trifluoroacetic acid, any of which may be present in an amount ranging from catalytic to a stoichiometric amount.

In other embodiments of the present invention is the method for preparing the compound of structural formula (VIII), wherein the protic acid is p-toluenesulfonic acid.

In other embodiments of the present invention is the method for preparing the compound of structural formula (VIII), wherein p-toluenesulfonic acid is present in catalytic amounts.

In other embodiments of the present invention is the method for preparing the compound of structural formula (VIII), wherein said solvent is selected from the group consisting of benzene, toluene, xylene, and dichloroethane; or any of these may be combined together and utilized as co-solvents.

In other embodiments of the present invention is the method for preparing the compound of structural formula (VIII), wherein said solvent is toluene.

In other embodiments of the present invention is the method for preparing the compound of structural formula (VIII), wherein the suitable temperature range is from about −25° C. to 140° C.

In other embodiments of the present invention is the method for preparing the compound of structural formula (VIII), wherein said suitable temperature range is from 70° C. to 90° C.

In other embodiments of the present invention is the method for preparing the compound of structural formula (VIII), wherein the appropriate reaction time range is from about 5 minutes to 24 hours.

In other embodiments of the present invention is the method for preparing the compound of structural formula (VIII), wherein said reaction time range is from 2 to 6 hours.

In yet other embodiments of the present invention is the method for preparing the compound of structural formula (IX):

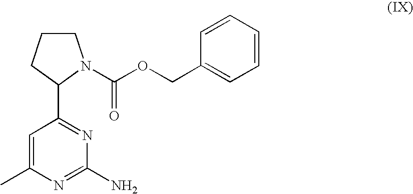

or a salt, ester, or prodrug thereof, comprising:
a) a treating the reaction product of structural formula (VIII),

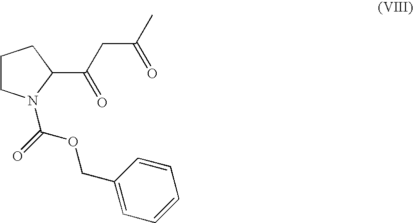

with suitable reagents including, but not limited to, an excess of an inorganic salt of guanidine, and an inorganic base, in a suitable solvent, using an appropriate reaction time over a suitable temperature range; and
b) optionally isolating the reaction product of structural formula (IX).

In yet other embodiments of the present invention is the method for preparing the compound of structural formula (IX), wherein said inorganic salt of guanidine is selected from the group consisting of guanidine hydrochloride, guanidine carbonate, and guanidine sulfate, any of which may be present in an amount greater than or equal to a stoichiometric amount.

In yet other embodiments of the present invention is the method for preparing the compound of structural formula (IX), wherein the inorganic salt is guanidine hydrochloride.

In yet other embodiments of the present invention is the method for preparing the compound of structural formula (IX), wherein said guanidine hydrochloride is present in greater than a stoichiometric amount.

In yet other embodiments of the present invention is the method for preparing the compound of structural formula (IX), wherein said solvent is selected from the group consisting of methanol, ethanol, 1-propanol, 2-propanol, 1-butanol, and tert-butanol; or any of these may be combined together and utilized as co-solvents.

In yet other embodiments of the present invention is the method for preparing the compound of structural formula (IX), wherein said solvent is ethanol.

In yet other embodiments of the present invention is the method for preparing the compound of structural formula (IX), wherein the suitable temperature range is from about 0° C. to 120° C.

In yet other embodiments of the present invention is the method for preparing the compound of structural formula (IX), wherein said suitable temperature range is from 70° C. to 90° C.

In yet other embodiments of the present invention is the method for preparing the compound of structural formula (IX), wherein the appropriate reaction time range is from about 5 minutes to 24 hours.

In yet other embodiments of the present invention is the method for preparing the compound of structural formula (IX), wherein said reaction time range is from 10 to 14 hours.

In other embodiments of the present invention is the method for preparing the compound of structural formula (X):

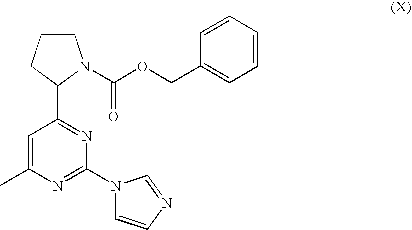

or a salt, ester, or prodrug thereof, comprising:
a) treating the reaction product of structural formula (IX),

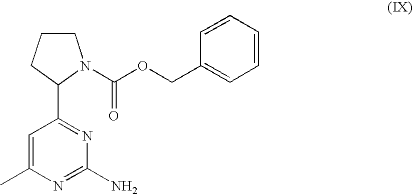

with a combination of suitable reagents, comprising a formaldehyde equivalent, a glyoxal equivalent, a ammonia equivalent, and an appropriate amount of a protic acid in a suitable protic solvent, using an appropriate reaction time over a suitable temperature range; and
b) isolating the novel reaction product of structural formula (X) in high yield and purity.

In other embodiments of the present invention is the method for preparing the compound of structural formula (X), wherein said formaldehyde equivalent is formalin or paraformaldehyde, either of which may be present in an amount greater than or equal to a stoichiometric amount.

In other embodiments of the present invention is the method for preparing the compound of structural formula (X), wherein paraformaldehyde is utilized as a reagent.

In other embodiments of the present invention is the method for preparing the compound of structural formula (X), wherein said paraformaldehyde is present in stoichiometric amounts.

In other embodiments of the present invention is the method for preparing the compound of structural formula (X), wherein said glyoxal equivalent is anhydrous glyoxal or glyoxal hydrate, either of which may be present in an amount greater than or equal to a stoichiometric amount.

In other embodiments of the present invention is the method for preparing the compound of structural formula (X), wherein glyoxal hydrate is employed as a reagent.

In other embodiments of the present invention is the method for preparing the compound of structural formula (X), wherein said glyoxal hydrate is present in stoichiometric amounts.

In other embodiments of the present invention is the method for preparing the compound of structural formula (X), wherein said ammonia equivalent is selected from the group consisting of ammonium chloride, ammonia gas, ammonium hydroxide, ammonium acetate, ammonium sulfate, ammonium bicarbonate, and ammonium carbamate, any of which may be present in an amount greater than or equal to a stoichiometric amount.

In other embodiments of the present invention is the method for preparing the compound of structural formula (X), wherein ammonium chloride is employed as a reagent.

In other embodiments of the present invention is the method for preparing the compound of structural formula (X), wherein said ammonium chloride is present in stoichiometric amounts. In other embodiments of the present invention is the method for preparing the compound of structural formula (X), wherein said protic acid is phosphoric acid, present in an amount ranging from catalytic to greater than or equal to a stoichiometric amount.

In other embodiments of the present invention is the method for preparing the compound of structural formula (X), wherein said phosphoric acid is present in stoichiometric amounts.

In other embodiments of the present invention is the method for preparing the compound of structural formula (X), wherein said phosphoric acid is present in catalytic amounts.

In other embodiments of the present invention is the method for preparing the compound of structural formula (X), wherein:

said solvent is selected from the group consisting of water, dioxane, ethanol, methanol, 1-propanol, 2-propanol, 1-butanol, tert-butanol, methoxyethanol, ethoxyethanol, ethylene glycol, and propylene glycol; or any of these protic solvents may be combined together and utilized as co-solvents.

In other embodiments of the present invention is the method for preparing the compound of structural formula (X), wherein dioxane and water are employed as the solvents.

In other embodiments of the present invention is the method for preparing the compound of structural formula (X), wherein the suitable temperature range is from about 0° C. to 150° C.

In other embodiments of the present invention is the method for preparing the compound of structural formula (X), wherein said suitable temperature range is from 80° C. to 110° C.

In other embodiments of the present invention is the method for preparing the compound of structural formula (X), wherein said reaction time range is from about 5 minutes to 48 hours.

In other embodiments of the present invention is the method for preparing the compound of structural formula (X), wherein said reaction time range is from 0.5 to 4 hours.

In yet other embodiments of the present invention is the method for preparing the compound of structural formula (XI):

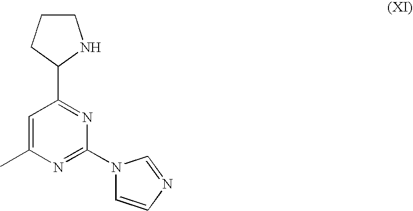

or a salt, ester, or prodrug thereof, comprising:
a) treating the reaction product of structural formula (X),

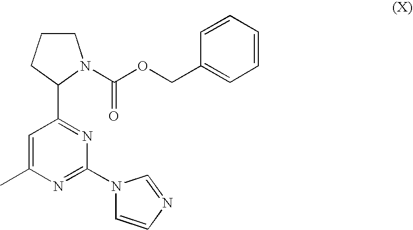

with a hydrogen source in the presence of a catalyst in a suitable protic solvent, under an appropriate pressure, using an appropriate reaction time over a suitable temperature range; and
b) isolating the novel reaction product of structural formula (XI) in high yield and purity.

In yet other embodiments of the present invention is the method for preparing the compound of structural formula (XI), wherein said hydrogen source is selected from the group consisting of hydrogen gas, cyclohexene in the presence of palladium on carbon, and ammonium formate in the presence palladium on carbon.

In yet other embodiments of the present invention is the method for preparing the compound of structural formula (XI), wherein hydrogen gas is employed as a reagent.

In yet other embodiments of the present invention is the method for preparing the compound of structural formula (XI), wherein said catalyst is selected from the group consisting of palladium on carbon, palladium hydroxide on carbon, platinum (IV) oxide, and platinum (IV) oxide on carbon; any of which may be present in an amount ranging from catalytic to greater than or equal to a stoichiometric amount.

In yet other embodiments of the present invention is the method for preparing the compound of structural formula (XI), wherein said catalyst is palladium on carbon.

In yet other embodiments of the present invention is the method for preparing the compound of structural formula (XI), wherein palladium on carbon is present in catalytic amounts.

In yet other embodiments of the present invention is the method for preparing the compound of structural formula (XI), wherein:

said protic solvent is selected from the group consisting of water, ethanol, methanol, 1-propanol, 2-propanol; or any of these solvents may be combined together and utilized as co-solvents.

In yet other embodiments of the present invention is the method for preparing the compound of structural formula (XI), wherein ethanol is employed as the solvent.

In yet other embodiments of the present invention is the method for preparing the compound of structural formula (XI), wherein the appropriate reaction pressure is selected from the range between one to four atmospheres.

In yet other embodiments of the present invention is the method for preparing the compound of structural formula (XI), wherein the reaction pressure is one atmosphere.

In yet other embodiments of the present invention is the method for preparing the compound of structural formula (XI), wherein the suitable temperature range is from about 0° C. to 100° C.

In yet other embodiments of the present invention is the method for preparing the compound of structural formula (XI), wherein said suitable temperature range is from 20° C. to 40° C.

In yet other embodiments of the present invention is the method for preparing the compound of structural formula (XI), wherein said reaction time range is from about 5 minutes to 48 hours.

In yet other embodiments of the present invention is the method for preparing the compound of structural formula (XI), wherein said reaction time range is from 0.5 to 6 hours.

In certain embodiments of the present invention is the method for preparing the compound of structural formula (XII):

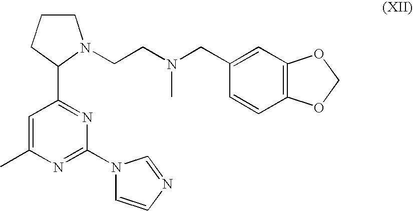

or a salt, ester, or prodrug thereof, comprising:
a) treating the reaction product of structural formula (XI),

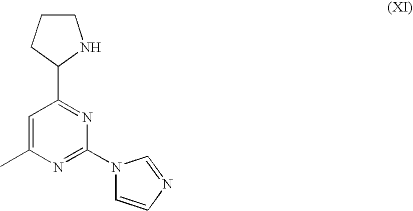

with the alkylating agent benzo[1,3]dioxol-5-ylmethyl-(2-chloro-ethyl)-methyl-amine hydrochloride salt, in the presence of a tertiary amine base and a halide salt in a suitable dipolar aprotic solvent using an appropriate reaction time over a suitable temperature range; and
b) isolating the novel reaction product of structural formula (XII) in high yield and purity.

In certain embodiments of the present invention is the method for preparing the compound of structural formula (XII), wherein:

said tertiary amine base is selected from the group consisting of triethylamine, N,N-diisopropylethylamine, 4-methylmorpholine, and N-methylpiperidine.

In certain embodiments of the present invention is the method for preparing the compound of structural formula (XII), wherein the tertiary amine base is N,N-diisopropylethylamine.

In certain embodiments of the present invention is the method for preparing the compound of structural formula (XII), wherein the halide salt is potassium iodide, which may be present in an amount ranging from catalytic to greater than or equal to a stoichiometric amount.

In certain embodiments of the present invention is the method for preparing the compound of structural formula (XII), wherein potassium iodide is present in catalytic amounts.

In certain embodiments of the present invention is the method for preparing the compound of structural formula (XII), wherein:

said suitable dipolar aprotic solvents are selected from the group consisting of dimethyl sulfoxide, sulfolane, N,N-dimethylformamide, N,N-dimethylacetamide, 1,3-dimethyl-3,4,5,6-tetrahydro-2(1H)-pyrimidinone, and hexamethylphosphoramide; or any of these may be combined together and utilized as co-solvents.

In certain embodiments of the present invention is the method for preparing the compound of structural formula (XII), wherein N,N-dimethylformamide is employed as the dipolar aprotic solvent.

In certain embodiments of the present invention is the method for preparing the compound of structural formula (XII), wherein the suitable temperature range is from about 0° C. to 150° C.

In certain embodiments of the present invention is the method for preparing the compound of structural formula (XII), wherein said suitable temperature range is from 70° C. to 90° C.

In certain embodiments of the present invention is the method for preparing the compound of structural formula (XII), wherein said reaction time range is from about 5 minutes to 48 hours.

In certain embodiments of the present invention is the method for preparing the compound of structural formula (XII), wherein said reaction time range is from 0.5 to 6 hours.

It should be recognized that certain modification to the process will be obvious to those skilled in the art, and such changes are intended to be within the scope of this invention. It is intended that the process will be carried out by skilled chemists who may make changes, such as preferably, but not necessarily, carrying out sequential reactions in the same vessel, or changing solvents or reaction temperatures or equipment, especially for economic reasons, and such modifications are to be considered within the scope of this invention.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The term “ambient temperature,” as used herein, alone or in combination, refers to the actual surrounding room temperature during the specified reaction period, and generally refers to a temperature range of about 20° C. to about 30° C., more preferably a temperature range of about 22° C. to about 27° C.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “ammonia equivalent,” as used herein, alone or in combination, refers to a reagent which serves as a synthetic equivalent of ammonia under the specified reaction conditions. Examples include ammonium chloride, ammonia gas, ammonium hydroxide, ammonium acetate, ammonium sulfate, ammonium bicarbonate, ammonium sulfate, and ammonium carbamate.

The term “aprotic solvent,” as used herein, alone or in combination, refers to a solvent which does not contain a hydrogen atom attached to a strongly electronegative element. It cannot donate a hydrogen atom to hydrogen bonding interactions. Examples of aprotic solvents include dichloromethane, toluene, acetone, ethyl acetate, diethyl ether, and tetrahydrofuran.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to C(O)OH or the corresponding “carboxylate” anion, such as is in a carboxylic acid salt. An “O-carboxy” group refers to a RC(O)O group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “dipolar aprotic solvent” as used herein, alone or in combination, refers to a polar solvent possessing a comparatively high relative permittivity (or dielectric constant), greater than ca. 15, and a sizable permanent dipole moment, that cannot donate suitably labile hydrogen atoms to form strong hydrogen bonds. Examples of dipolar aprotic solvents include dimethyl sulfoxide, N,N-dimethyformamide, 1,3-dimethyl-3,4,5,6-tetrahydro-2(1H)-pyrimidinone, hexamethylphosphoramide, acetonitrile, and the like.

The term “DCM” as used herein, alone or in combination, refers to dichloromethane.

The term “DMSO” as used herein, alone or in combination, refers to dimethyl sulfoxide.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “formaldehyde equivalent,” as used herein, alone or in combination, refers to a reagent which serves as a synthetic equivalent of formaldehyde under the specified reaction conditions. Examples include formaldehyde gas, formalin, formaldehyde sodium bisulfite addition product, paraformaldehyde, methylal, and s-1,3,5-trioxane.

The term “glyoxal equivalent,” as used herein, alone or in combination, refers to a reagent which serves as a synthetic equivalent of glyoxal under the specified reaction conditions. Examples include glyoxal, glyoxal hydrate, and glyoxal bis-sodium bisulfite addition product.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The phrase “in the main chain” refers to the longest contiguous or adjacent chain of carbon atoms starting at the point of attachment of a group to the compounds of this invention.

The term “lower,” as used herein, alone or in combination, means containing from 1 to and including 6 carbon atoms.

The term “N-alkylating agent,” as defined herein, refers to a combination of reagents capable of alkylating an amine group, such as an aldehyde with a combination of a reducing agent and reaction conditions capable of reducing an iminium compound, or to an alkyl halide or dialkylsulfate in the presence of a mild base, for example, a tertiary amine or an alkali metal carbonate.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “protic solvent” as used herein, alone or in combination, refers to a solvent that carries hydrogen attached to oxygen as in a hydroxyl group or attached to nitrogen as in an amine group. Such solvents can donate an H⁺ (proton). Examples of protic solvents include water, ethanol, tert-butanol, and diethylamine.

The term “protic acid” refers to those acids such as HCl, H₂SO₄, H₃PO₄, p-toluenesulfonic acid, trifluoroacetic acid, acetic acid, methane sulfonic acid, or a strongly acidic cationic ion exchange resin, such as Dowex® 50 or Amberlyte® IR-112, for example.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “TBME” refers to tert-butyl methyl ether.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —C₁₋₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

When a particular measure or amount is referred to, unless otherwise stated, said amount should be understood to be reasonably approximate, “reasonable” being subject to interpretation by one of skill in the art. Those of skill in the art regularly employ minor variations in quantitation to achieve ideal results. Furthermore, weights and measures are subject to variation based upon the sensitivity of the apparatus.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Thus, preferred salts include hydrochloride, hydrobromide, acetate, adipate, oxalate, phosphate, hippurate, L-ascorbate, benzenesulfonate (besylate), benzoate, citrate, fumarate, gentisate, glutarate, glycolate, 1-hydroxy-2-napthoate, p-hydroxybenzoate, maleate, L-malate, malonate, DL mandelate, methanesulfonate (mesylate), nicotinate, p-toluenesulfonate (tosylate), pyroglutamate, succinate, sulfate, L-(+)tartrate, and DL-tartarate salts of compounds of the present invention. A salt of a compound can be made by reacting the appropriate compound in the form of the free base with the appropriate acid.

### General Synthetic Methods for Preparing Compounds

The invention is further illustrated by the following examples.

### EXAMPLE 1

### 2-(2-(2-(1H-Imidazol-1-yl)-6-methylpyrimidin-4-yl)pyrrolidin-1-yl)-N-(benzo[d][1,3]dioxol-5-ylmethyl)-N-methylethanamine

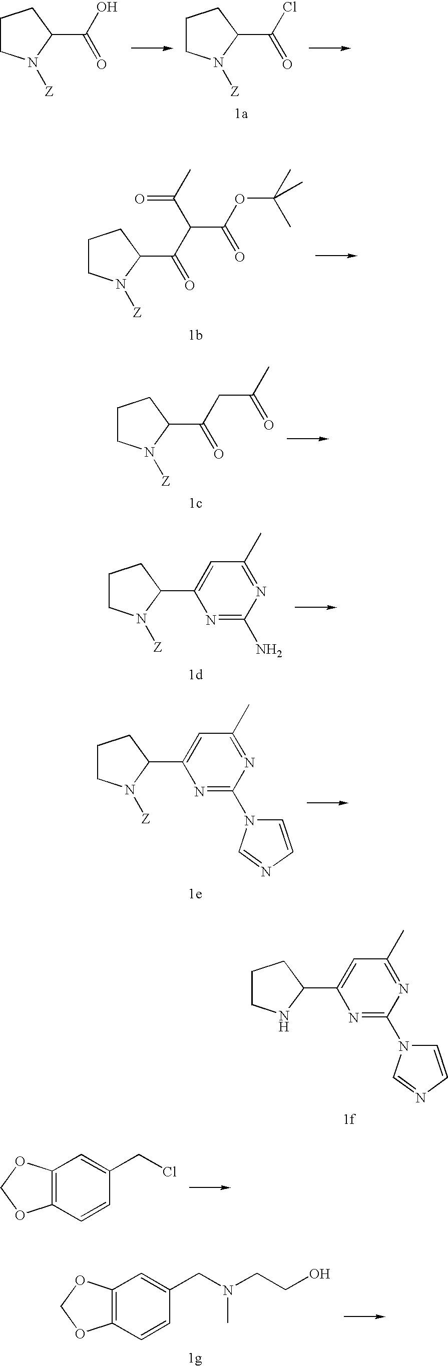

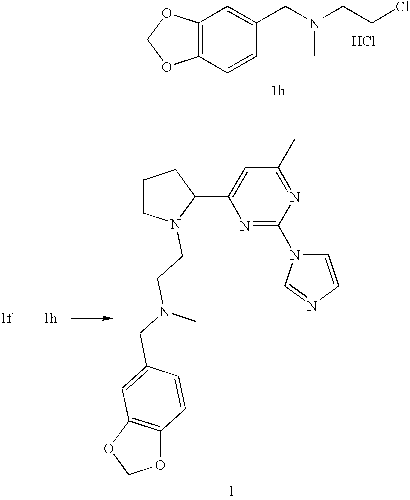

### Step 1:

### Preparation of compound 1a: 2-Chlorocarbonyl-pyrrolidine-1-carboxylic acid benzyl ester

Oxalyl chloride (707 g, 5.60 mol) was added dropwise (1 h) to a 3° C. solution of N-carbobenzyloxy-D,L-proline (1.00 kg, 4.01 mol), dimethylformamide (0.10 mL) and methylene chloride (4.00 L) under nitrogen. The mixture was warmed to room temperature and stirred for 14 h. The reaction mixture was concentrated to give 1.07 kg (100%) of 2-chlorocarbonyl-pyrrolidine-1-carboxylic acid benzyl ester as an amber oil.

### Step 2:

### Preparation of compound 1b: 2-(2-tert-Butoxycarbonyl-3-oxo-butyryl)-pyrrolidine-1-carboxylic acid benzyl ester

Methylmagnesium chloride (163 mL of a 3.00 M solution in THF, 489 mmol) was added dropwise to a 4° C. solution of tert-butylacetoacetate (79.0 g, 500 mmol) and THF (500 mL) while maintaining an internal temperature of 4-10° C. The reaction mixture was warmed to 15° C. and 2-chlorocarbonyl-pyrrolidine-1-carboxylic acid benzyl ester (66.0 g, 250 mmol) was added dropwise over 1 h. The mixture was warmed to room temperature and stirred for 12 h. NH₄Cl (300 mL of a saturated aqueous solution) was added and the phases were separated. The organic layer was concentrated under vacuum to give 97.4 g (100%) of 2-(2-tert-butoxycarbonyl-3-oxo-butyryl)-pyrrolidine-1-carboxylic acid benzyl ester as a yellow oil.

### Step 3:

### Preparation of compound 1c: 2-(3-Oxo-butyryl)-pyrrolidine-1-carboxylic acid benzyl ester

2-(2-tert-Butoxycarbonyl-3-oxo-butyryl)-pyrrolidine-1-carboxylic acid benzyl ester (97.4 g, 250 mmol) was dissolved toluene (400 mL) and was washed with 1N HCl (2×500 mL). p-Toluenesulfonic acid monohydrate (10.0 g, 50.0 mmol) was added to the organic layer and the solution was heated to 80° C. for 4 h under nitrogen. The mixture was cooled to room temperature and water (3×1 L) was added. The phases were separated and the organic layer was concentrated to give 68.7 g (95%) of 2-(3-oxo-butyryl)-pyrrolidine-1-carboxylic acid benzyl ester as an amber oil. [M+H]⁺ 290.03.

### Step 4:

### Preparation of compound 1d: 2-(2-Amino-6-methyl-pyrimidin-4-yl)-pyrrolidine-1-carboxylic acid benzyl ester

Sodium (5.50 g, 250 mmol) was added portionwise to a stirred solution of anhydrous ethanol (300 mL) under nitrogen at room temperature. A suspension of guanidine hydrochloride (22.8 g, 250 mmol) in ethanol (200 mL) was added and the resulting mixture was stirred for 20 minutes. The precipitate was removed by vacuum filtration and 2-(3-oxo-butyryl)-pyrrolidine-1-carboxylic acid benzyl ester (68.7 g, 237 mmol) was added to the filtrate. The solution was transferred to a flask fitted with a Dean-Stark trap and the reaction mixture was heated to 80° C. The solution was heated at 80° C. under nitrogen for 12 h while removing 200 mL of distillate. The mixture was allowed to cool to room temperature and was gradually cooled to −5° C. The resulting solid was collected by filtration and air dried to give 33.7 g (46%) of 2-(2-amino-6-methyl-pyrimidin-4-yl)-pyrrolidine-1-carboxylic acid benzyl ester as cream colored crystals. [M+H]⁺ 312.88.

### Step 5:

### Preparation of compound 1e: 2-(2-Imidazol-1-yl-6-methyl-pyrimidin-4-yl)-pyrrolidine-1-carboxylic acid benzyl ester

H₃PO₄ (470 μL) was added to a clear solution of 2-(2-amino-6-methyl-pyrimidin-4-yl)-pyrrolidine-1-carboxylic acid benzyl ester (2.65 g, 8.48 mmol), dioxane (31.2 mL) and water (4.24 mL) at room temperature to give a yellow suspension. Glyoxal (40 wt % in water, 1.23 g, 8.48 mmol), paraformaldehyde (254 mg, 8.48 mmol) and water (8.48 mL) were added and the suspension was heated to 80° C. Saturated NH₄Cl (453 mg, 8.48 mmol in 2.40 mL of H₂O) was added dropwise to the solution at 80° C. prior to heating at 100° C. for 2 h. The mixture was cooled to rt and bought to pH 12 with 4M NaOH then extracted with ethyl acetate. The combined organics were washed with brine and concentrated under vacuum. The product was purified by column chromatography (5:1 ethyl acetate/hexanes) to give 1.98 g (64%) of 2-(2-imidazol-1-yl-6-methyl-pyrimidin-4-yl)-pyrrolidine-1-carboxylic acid benzyl ester as a white solid. [M+H]⁺ 363.78.

### Step 6:

### Preparation of compound 1f: 2-Imidazol-1-yl-4-methyl-6-pyrrolidin-2-yl-pyrimidine

10% Pd/C (12 mg) was added to a solution of 2-(2-imidazol-1-yl-6-methyl-pyrimidin-4-yl)-pyrrolidine-1-carboxylic acid benzyl ester (112 mg, 0.308 mmol) and ethanol (3 mL) at room temperature. The solution was flushed with nitrogen then stirred under an atmosphere of hydrogen for 4 h. The reaction mixture was filtered through celite and concentrated under vacuum. The product was purified by column chromatography (DCM to 20% MeOH/DCM) to give 63 mg (89%) of 2-imidazol-1-yl-4-methyl-6-pyrrolidin-2-yl-pyrimidine. [M+H]⁺ 230.16; ¹H-NMR (400 MHz, CD₃OD) δ 8.74 (s, 1H), 8.05 (s, 1H), 7.31 (s, 1H), 7.14 (s, 1H), 4.95 (s, 2H), 4.25 (t, 1H), 3.25 (m, 1H), 3.05 (m, 1H), 2.59 (s, 3H), 2.35 (m, 1H), 1.90 (m, 2H); ¹³C-NMR (100 MHz, CD₃OD) δ 173.4, 170.4, 153.9, 136.0, 128.9, 116.8, 116.0, 62.1, 46.5, 32.7, 25.3, 22.7.

### Step 7:

### Preparation of compound 1g: 2-(Benzo[1,3]dioxol-5-ylmethyl-methyl-amino)-ethanol

2-(Methylamino)ethanol (22.0 g, 290 mmol) was added to a stirred solution of 3,4-methylenedioxybenzyl chloride (25.0 g, 147 mmol) in DCM (45 mL) at −78° C. under nitrogen. The solution was stirred for 15 minutes at −78° C. then warmed to room temperature and stirred for 16 h. 1.2 M NaOH (100 mL) was added and the phases were separated. The organic layer was washed water (2×150 mL) and concentrated under vacuum to give 25.3 g (83%) of 2-(benzo[1,3]dioxol-5-ylmethyl-methyl-amino)-ethanol as a clear oil.

### Step 8:

### Preparation of compound 1h: Benzo[1,3]dioxol-5-ylmethyl-(2-chloro-ethyl)-methyl-amine hydrochloride salt

Thionyl chloride (60 mL) was added dropwise over 30 minutes to a 0° C. solution of 2-(benzo[1,3]dioxol-5-ylmethyl-methyl-amino)-ethanol (22.2 g, 110 mmol) in DCM (250 mL) under nitrogen. The solution was warmed to room temperature and stirred for 16 h. The suspension was concentrated under vacuum and brine (150 mL) and ethyl acetate (200 mL) were added. The precipitate was collected by vacuum filtration and washed with ethyl acetate (100 mL). The solid was dried overnight under vacuum to give 26.5 g (91%) of benzo[1,3]dioxol-5-ylmethyl-(2-chloro-ethyl)-methyl-amine hydrochloride as a white powder.

### Step 9:

### Preparation of compound 1: 2-(2-(2-(1H-Imidazol-1-yl)-6-methylpyrimidin-4-yl)pyrrolidin-1-yl)-N-(benzo[d][1,3]dioxol-5-ylmethyl)-N-methylethanamine

A solution of 2-imidazol-1-yl-4-methyl-6-pyrrolidin-2-yl-pyrimidine (2.1 g, 9.2 mmol) in DMF (15 mL) was added to a stirred mixture of benzo[1,3]dioxol-5-ylmethyl-(2-chloro-ethyl)-methyl-amine hydrochloride salt (2.2 g, 8.1 mmol), DMF (10 mL) and diisopropylethylamine (2.5 mL) at room temperature under nitrogen. Potassium iodide (340 mg, 2.0 mmol) was added and the mixture was heated to 80° C. for 3 h. The solution was cooled to room temperature and 1N dibasic potassium phosphate solution (200 mL) was added. The solution was extracted with ethyl acetate and the phases were separated. The organic layer was concentrated and the product was purified by column chromatography (DCM to 4:1 DCM/MeOH) to give 2.0 g (52%) of 2-(2-(2-(1H-imidazol-1-yl)-6-methylpyrimidin-4-yl)pyrrolidin-1-yl)-N-(benzo[d][1,3]dioxol-5-ylmethyl)-N-methylethanamine as a red oil. [M+H]⁺ 421.30; ¹H-NMR (400 MHz, CDCl₃) δ 8.60 (s, 1H), 7.89 (s, 1H), 7.30 (s, 1H), 7.10 (s, 1H), 6.78 (s, 1H), 6.67 (m, 2H), 5.88 (s, 2H), 3.52 (t, 1H), 3.6 (m, 3H), 2.77 (m, 1H), 2.2-2.6 (m, 8H), 2.35 (s, 3H), 1.62-1.95 (m, 3H); ¹³C-NMR (100 MHz, CDCl₃) δ 175.7, 169.6, 154.0, 147.6, 146.5, 136.2, 132.8, 130.1, 121.9, 116.6, 115.0, 109.2, 107.8, 100.8, 69.8, 62.3, 56.0, 54.3, 53.1, 42.5, 33.2, 24.2, 23.4.

### EXAMPLE 2

### 2-(2-(2-(1H-Imidazol-1-yl)-6-methylpyrimidin-4-yl)pyrrolidin-1-yl)-N-(benzo[d][1,3]dioxol-5-ylmethyl)-N-methylethanamine (Enantiomer 1)

Compound 2 was prepared following the procedures described in preparation of Example 1. A single enantiomer of Example 1 was obtained by chiral HPLC (chiralpak ADRH, 4.6×150 mm, 10 mM NH₄OAc/EtOH 4:6 (v/v), flow rate 0.5 mL/min) separation. Analytical data are identical to Example 1.

### EXAMPLE 3

### 2-(2-(2-(1H-Imidazol-1-yl)-6-methylpyrimidin-4-yl)pyrrolidin-1-yl)-N-(benzo[d][1,3]dioxol-5-ylmethyl)-N-methylethanamine (Enantiomer 2)

Compound 3 was prepared following the procedures described in preparation of Example 1. A single enantiomer of Example 1 was obtained by chiral HPLC (chiralpak ADRH, 4.6×150 mm, 10 mM NH₄OAc/EtOH 4:6 (v/v), flow rate 0.5 mL/min) separation. Analytical data are identical to Example 1.

### EXAMPLE 4

### 2-(2-(2-(1H-Imidazol-1-yl)-6-methylpyrimidin-4-yl)pyrrolidin-1-yl)-N-(benzo[d][1,3]dioxol-5-ylmethyl)-N-methylethanamine hydrochloric acid salt

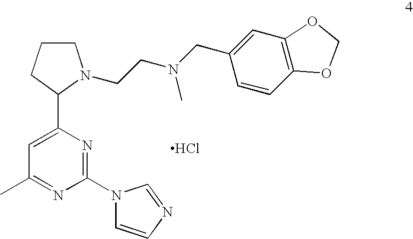

Hydrochloric acid (1.60 mL of a 12.39 M aqueous solution, 19.8 mmol) was added to a stirred solution of 1 (8.2 g, 19.5 mmol) in acetone (100 mL) at room temperature under nitrogen. The mixture was stirred for 1 h and the precipitate was collected by vacuum filtration. The solid was washed with acetone (30 mL) and dried under vacuum for 16 h to give 6.3 g (71%) of benzo[1,3]dioxol-5-ylmethyl-{2-[2-(2-imidazol-1-yl-6-methyl-pyrimidin-4-yl)-pyrrolidin-1-yl]-ethyl}-amine hydrochloric acid salt as a white solid. [M+H]⁺ 421.30; ¹H-NMR (400 MHz, DMSO) δ 8.54 (s, 1H), 7.90 (s, 1H), 7.49 (s, 1H), 7.11 (s, 1H), 7.08 (s, 1H), 6.90 (s, 2H), 6.02 (s, 2H), 4.09 (s, 2H), 3.70 (t, 1H), 3.50-3.20 (m, 3H), 3.10-2.90 (m, 3H), 2.54 (s, 3H), 2.52 (s, 3H), 2.46-2.40 (m, 1H), 2.30-2.20 (m, 1H), 1.94-1.82 (m, 2H), 1.80-1.70 (m, 1H).

### EXAMPLE 5

### 2-(2-Imidazol-1-yl-6-methyl-pyrimidin-4-yl)-pyrrolidine-1-carboxylic acid (2-benzo[1,3]dioxol-5-yl-ethyl)-amide

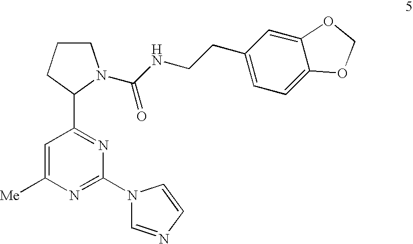

A solution of 2-imidazol-1-yl-4-methyl-6-pyrrolidin-2-yl-pyrimidine (21.8 mg, 0.095 mmol), 3,4-methylenedioxyphenethyl isocyanate (29 mg, 0.151 mmol), triethylamine (0.4 mL) and THF (1.5 mL) was stirred at room temperature under nitrogen for 10 minutes. Water was added and the mixture was extracted with ethyl acetate (2×3 mL). The combined organic layers were washed with brine and concentrated under vacuum. The product was purified by Prep-TLC to give 36 mg (90%) of 2-(2-imidazol-1-yl-6-methyl-pyrimidin-4-yl)-pyrrolidine-1-carboxylic acid (2-benzo[1,3]dioxol-5-yl-ethyl)-amide as a white solid. [M+H]⁺ 421.15; ¹H-NMR (400 MHz, CD₃OD) δ 8.65 (s, 1H), 7.99 (s, 1H), 7.14 (s, 1H), 7.11 (s, 1H), 6.65 (m, 3H), 5.88 (s, 2H), 4.98 (m, 1H), 3.62 (m, 1H), 3.55 (m, 1H), 3.34 (m, 2H), 2.69 (m, 2H), 2.56 (s, 3H), 2.41 (m, 1H), 2.03 (m, 3H); ¹³C-NMR (100 MHz, CD₃OD) δ 173.9, 170.3, 166.5, 157.7, 147.6, 145.9, 133.2, 128.9, 121.3, 115.0, 108.6, 107.6, 106.9, 61.4, 46.4, 41.8, 35.7, 32.4, 23.3, 22.8.

### EXAMPLE 6

### 1-(2-(2-(1H-Imidazol-1-yl)-6-methylpyrimidin-4-yl)pyrrolidin-1-yl)-2-(benzo[d][1,3]dioxol-5-ylmethylamino)ethanone

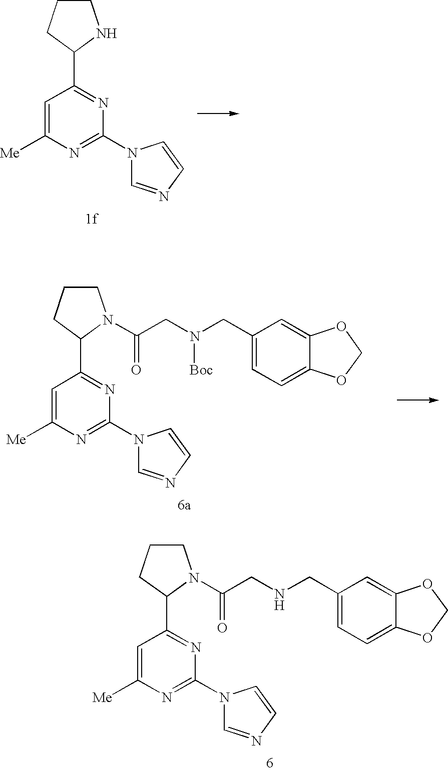

### Step 1:

### Preparation of compound 6a: tert-Butyl 2-(2-(2-(1H-imidazol-1-yl)-6-methylpyrimidin-4-yl)pyrrolidin-1-yl)-2-oxoethyl(benzo[d][1,3]dioxol-5-ylmethyl)carbamate

A solution of 1f (21.0 mg, 0.092 mmol), N-Boc-[(benzo[1,3]dioxaol-5-ylmethyl)amino]acetic acid (39 mg, 0.13=mol), 1-ethyl-3-(3-dimethylaminopropyl)carbodiimide hydrochloride (28 mg, 0.15 mmol) and 1-hydroxybenzotriazole (20 mg, 0.15 mmol) in DMF (1.5 mL) was stirred at room temperature under nitrogen for 30 min. Water was added and the mixture was extracted with ethyl acetate (2×10 mL). The combined organic layers were washed with brine and concentrated under vacuum. The product was purified by Prep-TLC to give 48 mg (100%) of tert-butyl 2-(2-(2-(1H-imidazol-1-yl)-6-methylpyrimidin-4-yl)pyrrolidin-1-yl)-2-oxoethyl(benzo[d][1,3]dioxol-5-ylmethyl)carbamate as a white solid.

### Step 2:

### Preparation of compound 6: 1-(2-(2-(1H-imidazol-1-yl)-6-methylpyrimidin-4-yl)pyrrolidin-1-yl)-2-(benzo[d][1,3]dioxol-5-ylmethylamino)ethanone

A mixture of 6a (48 mg, 0.092 mmol), TFA (0.5 mL) and DCM (0.5 mL) was stirred at room temperature under nitrogen for 20 min. The solution was concentrated under vacuum and Na₂CO₃ (10 mL) was added. The mixture was extracted with ethyl acetate (2×5 mL) and the combined organic layers were washed with brine (10 mL). The solution was concentrated under vacuum to give 25 mg (65%) of 2-(2imidazol-1-yl-6-methyl-pyrimidin-4-yl)-pyrrolidine-1-carboxylic acid (benzo[1,3]dioxol-5-ylmethyl)-amide as a white solid. [M+H]⁺ 421.07; ¹H-NMR (400 MHz, CD₃OD) δ 8.67 (s, 1H), 8.00 (s, 1H), 7.23 (s, 1H), 6.86 (s, 1H), 6.78 (m, 2H), 6.53 (s, 1H), 5.93 (s, 1H), 3.83 (m, 1H), 3.70-3.50 (m, 6H), 3.34 (s, 1H), 2.58 (s, 3H), 2.50-1.90 (m, 4H).

### EXAMPLE 7

### N-Benzo[1,3]dioxol-5-ylmethyl-2-[2-(2-imidazol-1-yl-6-methyl-pyrimidin-4-yl)-pyrrolidin-1-yl]-acetamide

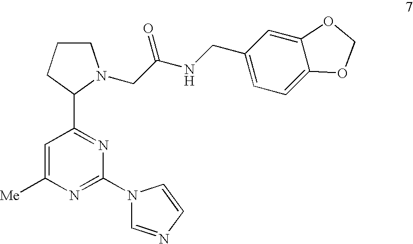

A solution of 1f (4 mg, 0.02 mmol), N-benzo[1,3]dioxol-5-ylmethyl-2-chloro-acetamide (4 mg, 0.02 mmol), DMF (0.5 mL) and TEA (0.2 mL) was heated at 60° C. under nitrogen for 20 h. Water was added and the mixture was extracted with ethyl acetate. The organic layer was washed with brine and concentrated under vacuum. The product was purified by Prep-TLC to give 5 mg (60%) of N-benzo[1,3]dioxol-5-ylmethyl-2-[2-(2-imidazol-1-yl-6-methyl-pyrimidin-4-yl)-pyrrolidin-1-yl]-acetamide. [M+H]⁺ 421.09.

### EXAMPLE 8

### Benzo[1,3]dioxol-5-ylmethyl-{2-[2-(2-imidazol-1-yl-6-methyl-pyrimidin-4-yl)-pyrrolidin-1-yl]-ethyl}-amine

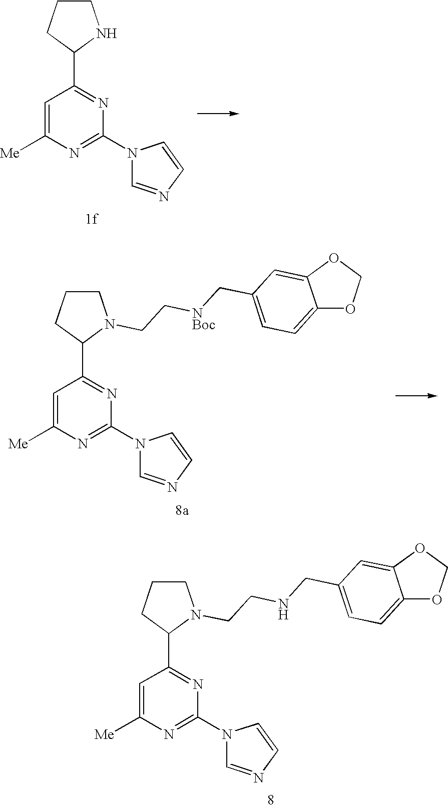

### Step 1:

Preparation of compound 8a: Benzo[1,3]dioxol-5-ylmethyl-{2-[2-(2-imidazol-1-yl-6-methyl-pyrimidin-4-yl)-pyrrolidin-1-yl]-ethyl}-carbamic acid tert-butyl ester was prepared following the procedures described in the preparation of Example 1 using tert-butyl benzo[d][1,3]dioxol-5-ylmethyl(2-bromoethyl)carbamate and 1f

### Step 2:

Preparation of compound 8: Benzo[1,3]dioxol-5-ylmethyl-{2-[2-(2-imidazol-1-yl-6-methyl-pyrimidin-4-yl)-pyrrolidin-1-yl]-ethyl}-amine was prepared following the procedures described in the preparation of Example 6 using benzo[1,3]dioxol-5-ylmethyl-{2-[2-(2-imidazol-1-yl-6-methyl-pyrimidin-4-yl)-pyrrolidin-1-yl]-ethyl}-carbamic acid tert-butyl ester. [M+H]⁺ 406.97; ¹H-NMR (400 MHz, CDCl₃) δ 8.54 (s, 1H), 7.83 (s, 1H), 7.19 (s, 1H), 7.04 (s, 1H), 6.71 (s, 1H), 6.64 (m, 2H), 5.85 (s, 2H), 3.60-3.20 (m, 5H), 2.80-2.20 (m, 9H), 1.90-1.60 (m, 2H); ¹³C-NMR (100 MHz, CDCl₃) δ 175.6, 170.0, 154.2, 147.9, 146.8, 136.4, 133.7, 130.2, 121.4, 116.9, 115.4, 108.7, 108.2, 101.1, 69.9, 54.6, 54.2, 53.7, 50.2, 47.6, 33.6, 24.4, 23.7.

### EXAMPLE 9

### (2,3-Dihydro-benzo[1,4]dioxin-6-ylmethyl)-{2-[2-(2-imidazol-1-yl-6-methyl-pyrimidin-4-yl)-pyrrolidin-1-yl]-ethyl}-amine

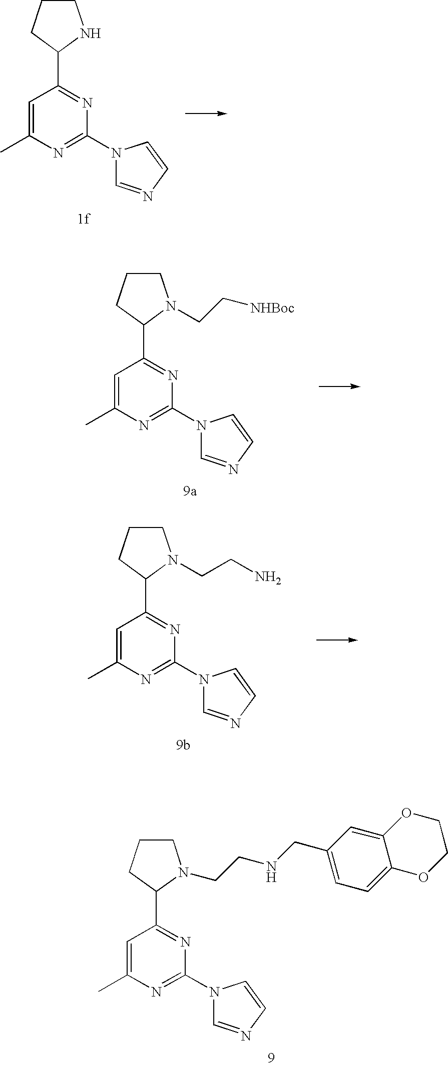

### Step 1:

Preparation of compound 9a: tert-Butyl 2-(2-(2-(1H-imidazol-1-yl)-6-methylpyrimidin-4-yl)pyrrolidin-1-yl)ethylcarbamate was prepared following the procedures described in the preparation of Example 1 using (2-bromo-ethyl)-carbamic acid tert-butyl ester and 1f. [M+H]⁺ 373.47.

### Step 2:

Preparation of compound 9b: 2-[2-(2-Imidazol-1-yl-6-methyl-pyrimidin-4-yl)-pyrrolidin-1-yl]-ethylamine was prepared following the procedures described in the preparation of Example 6 using {2-[2-(2-imidazol-1-yl-6-methyl-pyrimidin-4-yl)-pyrrolidin-1-yl]-ethyl}-carbamic acid tert-butyl ester. [M+H]⁺ 273.81.

### Step 3:

### Preparation of compound 9: (2,3-Dihydro-benzo[1,4]dioxin-6-ylmethyl)-{2-[2-(2-imidazol-1-yl-6-methyl-pyrimidin-4-yl)-pyrrolidin-1-yl]-ethyl}-amine

A solution of 9b (78 mg, 290 μmol), 2,3-dihydro-benzo[1,4]dioxine-6-carbaldehyde (47 mg, 290 μmol), p-toluenesulfonic acid monohydrate (5.0 mg, 26 μmol) and dioxane (3 mL) was heated at 60° C. for 16 h. The reaction mixture was cooled to room temperature and sodium triacetoxyborohydride (180 mg, 860 μmol) was added. The suspension was stirred at room temperature for 1 h prior to the addition of EtOAc (25 mL) and 1N NaOH (25 mL). The phases were separated and the organic layer was concentrated under vacuum. The product was purified using column chromatography (0% to 10% MeOH/DCM) to give 69 mg (57%) of (2,3-dihydro-benzo[1,4]dioxin-6-ylmethyl)-{2-[2-(2-imidazol-1-yl-6-methyl-pyrimidin-4-yl)-pyrrolidin-1-yl]-ethyl}-amine. [M+H]⁺ 421.23; ¹H NMR (400 MHz, CDCl₃) δ 8.60 (s, 1H), 7.89 (s, 1H), 7.23 (s, 1H), 7.12 (s, 1H), 6.68-6.79 (m, 3H), 4.21 (s, 4H), 3.54 (t, 1H), 3.22 (m, 1H), 2.70-2.82 (m, 2H), 2.20-2.60 (m, 9H), 1.84 (m, 2H), 1.70 (m, 1H).

### EXAMPLE 10

### (2-Benzo[1,3]dioxol-5-yl-ethyl)-{2-[2-(2-imidazol-1-yl-6-methyl-pyrimidin-4-yl)-pyrrolidin-1-yl]-ethyl}-amine

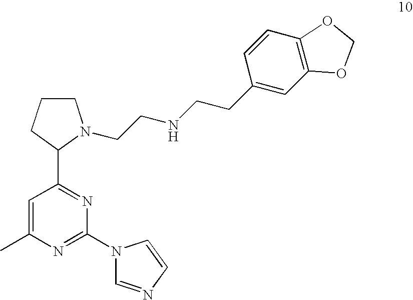

Compound 10 was prepared following the procedures described in the preparation of Example 9 using benzo[1,3]dioxol-5-yl-acetaldehyde and 2-[2-(2-imidazol-1-yl-6-methyl-pyrimidin-4-yl)-pyrrolidin-1-yl]-ethylamine. [M+H]⁺ 421.55; ¹H NMR (400 MHz, CDCl₃) δ 8.57 (s, 1H), 7.86 (s, 1H), 7.17 (s, 1H), 7.10 (s, 1H), 6.90 (d, 1H), 6.65 (d, 1H), 6.60 (dd, 1H), 5.88 (s, 2H), 3.50 (t, 1H), 3.21 (m, 1H), 2.82-2.64 (m, 5H), 2.50 (s, 3H), 2.40-2.20 (m, 4H), 1.95-1.66 (m, 4H).

### EXAMPLE 11

### [2-(2,3-Dihydro-benzo[1,4]dioxin-6-yl)-ethyl]-{2-[2-(2-imidazol-1-yl-6-methyl-pyrimidin-4-yl)-pyrrolidin-1-yl]-ethyl}-amine

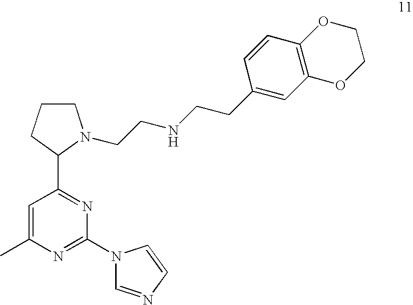

Compound 11 was prepared following the procedures described in the preparation of Example 9 using (2,3-dihydro-benzo[1,4]dioxin-6-yl)-acetaldehyde and 2-[2-(2-imidazol-1-yl-6-methyl-pyrimidin-4-yl)-pyrrolidin-1-yl]-ethylamine. [M+H]⁺ 435.54; ¹H NMR (400 MHz, CDCl₃) δ 8.59 (s, 1H), 7.85 (s, 1H), 7.15 (s, 1H), 7.10 (s, 1H), 6.75 (d, 1H), 6.70-6.60 (m, 2H), 4.18 (s, 4H), 3.54 (t, 1H), 3.15 (m, 1H), 2.94-2.74 (m, 5H), 2.52 (s, 3H), 2.40-2.20 (m, 4H), 1.95-1.66 (m, 4H).

### EXAMPLE 12

### (2,3-Dihydro-benzo[1,4]dioxin-6-ylmethyl)-{2-[2-(2-imidazol-1-yl-6-methyl-pyrimidin-4-yl)-pyrrolidin-1-yl]-ethyl}-methyl-amine

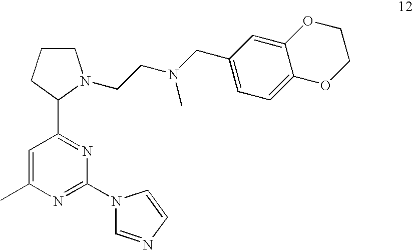

A solution of (2,3-dihydro-benzo[1,4]dioxin-6-ylmethyl)-{2-[2-(2-imidazol-1-yl-6-methyl-pyrimidin-4-yl)-pyrrolidin-1-yl]-ethyl}-amine (65 mg, 160 μmol), formalin (63 μL, 780 μmol), acetic acid (170 μL, 2.8 mmol) and MeOH (1 mL) was stirred at room temperature for 5 min. Sodium triacetoxyborohydride (99 mg, 470 μmol) was added and the mixture was stirred for 20 min. The solution was concentrated and EtOAc (5 mL) and 1N NaOH (5 mL) were added. The phases were separated and the organic layer was concentrated under vacuum. The product was purified using column chromatography (0% to 10% MeOH/DCM) to give 60 mg (89%) of (2,3-dihydro-benzo[1,4]dioxin-6-ylmethyl)-{2-[2-(2-imidazol-1-yl-6-methyl-pyrimidin-4-yl)-pyrrolidin-1-yl]-ethyl}-methyl-amine as a white solid. [M+H]⁺ 435.32; ¹H NMR (400 MHz, CDCl₃) δ 8.61 (s, 1H), 7.88 (s, 1H), 7.33 (s, 1H), 7.12 (s, 1H), 6.65-6.79 (m, 3H), 4.21 (s, 4H), 3.54 (t, 1H), 3.24-3.42 (m, 3H), 2.76 (m, 1H), 2.11-2.55 (m, 8H), 2.15 (s, 3H), 1.84 (m, 2H), 1.70 (m, 1H).

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method for the preparation of a compound of structural formula (VI): 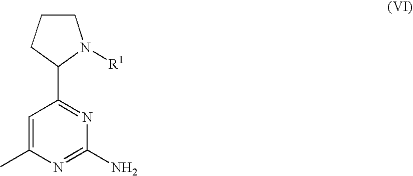
or a salt, ester, or prodrug thereof, wherein:
R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylarylalkylsulfonyl, heteroarylalkenylsulfonyl, heteroalkyl, heterocycloalkyl, hydroxyalkyl, perhaloalkyl, and trisubstituted silyl, any of which may be optionally substituted;
comprising:
a) treating the reaction product of structural formula (V), 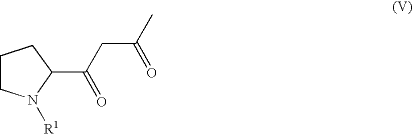
with suitable reagents including, but not limited to, an excess of an inorganic salt of guanidine, and an inorganic base, in a suitable protic solvent, using an appropriate reaction time over a suitable temperature range; and
b) optionally isolating the reaction product of structural formula (VI).

2. The method as recited in claim 1, wherein the inorganic salt is guanidine hydrochloride, present in greater than a stoichiometric amount.

3. The method as recited in claim 1, wherein said protic solvent is ethanol, and wherein the suitable temperature range is from 70° C. to 90° C.

4. The method as recited in claim 1, wherein said reaction time range is from 10 to 14 hours.

5. A method for the preparation of a compound of structural formula (I): 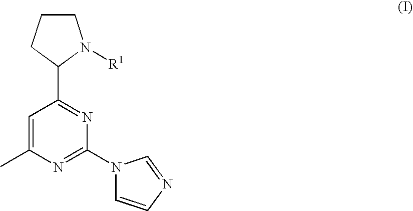
or a salt, ester, or prodrug thereof, wherein:
R¹ is selected from the group consisting of hydrogen, acyl, alkanoyl, alkenyl, alkenyloxycarbonyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboarylalkoxy, carboaryloxy, carboarylalkenyloxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted;
comprising:
a) treating the reaction product of structural formula (VI), 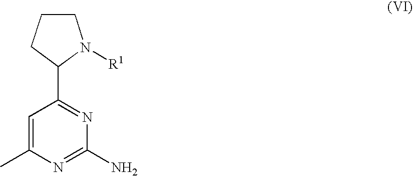
with a combination of suitable reagents, comprising a formaldehyde equivalent, a glyoxal equivalent, a ammonia equivalent, and an appropriate amount of a protic acid in a suitable protic solvent, using an appropriate reaction time over a suitable temperature range;
b) isolating the novel reaction product of structural formula (I in high yield and purity;
c) optionally removing the R¹ group to afford a compound of structural formula (I), wherein R¹ is hydrogen; and
d) optionally alkylating the compound of structural formula (I), wherein R¹ is hydrogen, with an appropriate alkylating agent in a suitable solvent, using an appropriate reaction time over a suitable temperature range.

6. The method as recited in claim 5, wherein paraformaldehyde is utilized as a reagent, and is present in stoichiometric amounts.

7. The method as recited in claim 5, wherein said glyoxal equivalent is glyoxal hydrate, and is present in stoichiometric amounts.

8. The method as recited in claim 5, wherein said ammonia equivalent is ammonium chloride and is present in stoichiometric amounts.

9. The method as recited in claim 5, wherein said protic acid is phosphoric acid, present in an amount ranging from catalytic to greater than or equal to a stoichiometric amount.

10. The method as recited in claim 9, wherein said phosphoric acid is present in catalytic amounts.

11. The method as recited in claim 5, wherein dioxane and water are employed as the solvents, and said suitable temperature range is from 80° C. to 110° C.

12. The method as recited in claim 5, wherein said reaction time range is from 0.5 to 4 hours.

13. A method for the preparation of a compound of structural formula (VII): 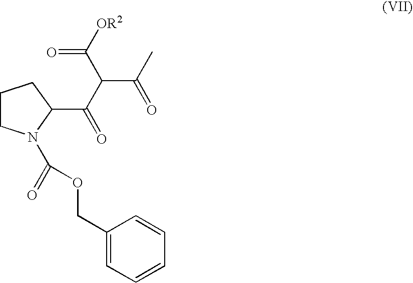
or a salt, ester, or prodrug thereof, wherein:
R² is optionally substituted alkyl;
comprising:
a) treating the reaction product of structural formula (IIIa), 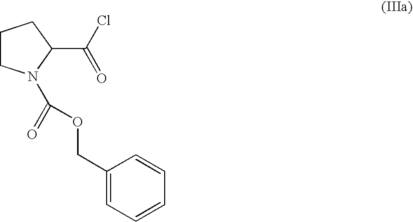
with an excess of the salt of a monoanion of alkylacetoacetate, in a suitable solvent, using an appropriate reaction time over a suitable temperature range reagents; and
b) isolating the novel reaction product of structural formula (VII).

14. The method as recited in claim 13, wherein the salt is the magnesium salt and said solvent is tetrahydrofuran.

15. The method as recited in claim 13, wherein said suitable temperature range is from 0° C. to ambient temperature.

16. The method as recited in claim 13, wherein said reaction time range is from 10 to 14 hours.

17. A method for the preparation of a compound of structural formula (VIII): 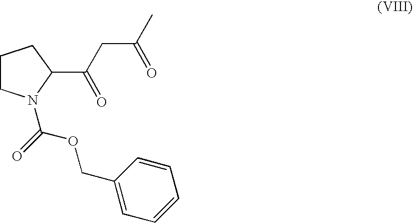
or a salt, ester, or prodrug thereof, comprising:
a) treating the reaction product of structural formula (VII), 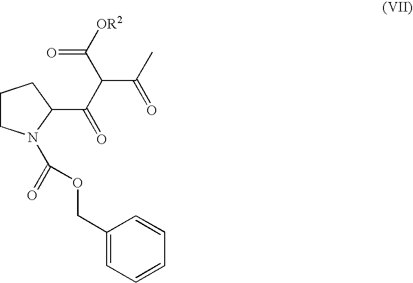
with a protic acid, in a suitable solvent, using an appropriate reaction time over a suitable temperature range; and in a suitable solvent; and
a) isolating the novel reaction product of structural formula (VIII).

18. The method as recited in claim 17, wherein said protic acid is p-toluenesulfonic acid, which may be present in an amount ranging from catalytic to a stoichiometric amount.

19. The method as recited in claim 19, wherein p-toluenesulfonic acid is present in catalytic amounts.

20. The method as recited in claim 17, wherein said solvent is toluene, and wherein said suitable temperature range is from 70° C. to 90° C.

21. The method as recited in claim 17, wherein said reaction time range is from 2 to 6 hours.

22. A method for the preparation of a compound of structural formula (IX): 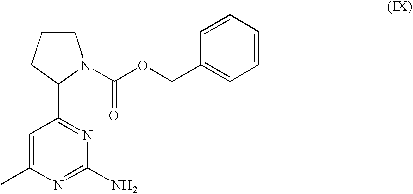
or a salt, ester, or prodrug thereof, comprising:
a) treating the reaction product of structural formula (VIII), 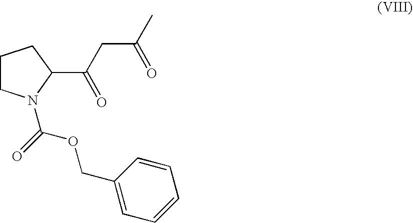
with suitable reagents including, but not limited to, an excess of an inorganic salt of guanidine, and an inorganic base, in a suitable protic solvent, using an appropriate reaction time over a suitable temperature range; and
b) optionally isolating the reaction product of structural formula (IX).

23. The method as recited in claim 22, wherein said inorganic salt of guanidine is guanidine hydrochloride, which may be present in an amount greater than or equal to a stoichiometric amount.

24. The method as recited in claim 23, wherein said guanidine hydrochloride is present in greater than a stoichiometric amount.

25. The method as recited in claim 22, wherein said protic solvent is ethanol and the suitable temperature range is 70° C. to 90° C.

26. The method as recited in claim 22, wherein said reaction time range is from 10 to 14 hours.

27. A method for the preparation of a compound of structural formula (X): 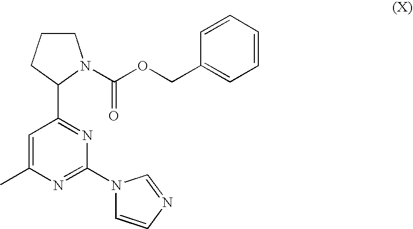
or a salt, ester, or prodrug thereof, comprising:
a) treating the reaction product of structural formula (IX), 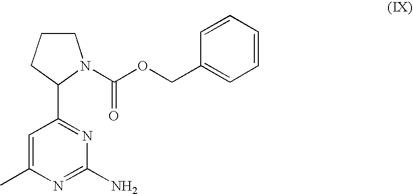
with a combination of suitable reagents, comprising a formaldehyde equivalent, a glyoxal equivalent, a ammonia equivalent, and an appropriate amount of a protic acid in a suitable solvent, using an appropriate reaction time over a suitable temperature range; and
b) isolating the novel reaction product of structural formula (X) in high yield and purity.

28. The method as recited in claim 27, wherein said formaldehyde equivalent is formalin or paraformaldehyde, either of which may be present in an amount greater than or equal to a stoichiometric amount.

29. The method as recited in claim 28, wherein said paraformaldehyde is present in stoichiometric amounts.

30. The method as recited in claim 27, wherein said glyoxal equivalent is glyoxal hydrate, which may be present in an amount greater than or equal to a stoichiometric amount.

31. The method as recited in claim 30, wherein said glyoxal hydrate is present in stoichiometric amounts.

32. The method as recited in claim 27, wherein said ammonia equivalent is ammonium chloride, which may be present in an amount greater than or equal to a stoichiometric amount.

33. The method as recited in claim 32, wherein said ammonium chloride is present in stoichiometric amounts.

34. The method as recited in claim 27, wherein said protic acid is phosphoric acid, present in an amount ranging from catalytic to greater than or equal to a stoichiometric amount.

35. The method as recited in claim 34, wherein said phosphoric acid is present in catalytic amounts.

36. The method as recited in claim 27, wherein said solvents are dioxane and water.

37. The method as recited in claim 27, wherein said suitable temperature range is from 80° C. to 110° C.

38. The method as recited in claim 27, wherein said reaction time range is from 0.5 to 4 hours.

39. A method for the preparation of a compound of structural formula (XI): 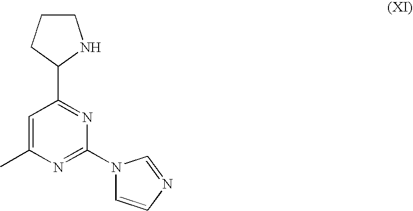
or a salt, ester, or prodrug thereof, comprising:
a) treating the reaction product of structural formula (X), 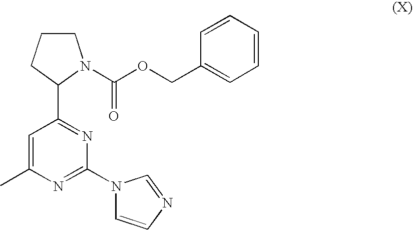
with a hydrogen source in the presence of a catalyst in a suitable protic solvent, under an appropriate reaction pressure, using an appropriate reaction time over a suitable temperature range; and
b) isolating the novel reaction product of structural formula (XI) in high yield and purity.

40. The method as recited in claim 39, wherein said hydrogen source is hydrogen gas.

41. The method as recited in claim 39, wherein said catalyst is palladium on carbon, present in catalytic amounts.

42. The method as recited in claim 39, wherein said protic solvent is ethanol.

43. The method as recited in claim 39, wherein the appropriate reaction pressure is one atmosphere, and said suitable temperature range is from 20° C. to 40° C.

44. The method as recited in claim 39, wherein said reaction time range is from 0.5 to 6 hours.

45. A method for the preparation of a compound of structural formula (XII): 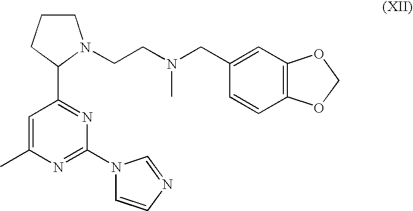
or a salt, ester, or prodrug thereof, comprising:
a) treating the reaction product of structural formula (XI), 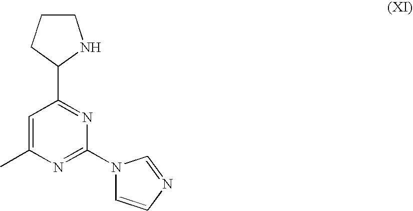
with the alkylating agent benzo[1,3]dioxol-5-ylmethyl-(2-chloro-ethyl)-methyl-amine hydrochloride salt, in the presence of a tertiary amine base and a halide salt in a suitable dipolar aprotic solvent using an appropriate reaction time over a suitable temperature range; and
b) isolating the novel reaction product of structural formula (XII) in high yield and purity.

46. The method as recited in claim 46, wherein said tertiary amine base is N,N-diisopropylethylamine.

47. The method as recited in claim 46, wherein the halide salt is potassium iodide and is present in catalytic amounts.

48. The method as recited in claim 46, wherein said suitable dipolar aprotic solvent is N,N-dimethylformamide.

49. The method as recited in claim 46, wherein said suitable temperature range is from 70° C. to 90° C.

50. The method as recited in claim 46, wherein said reaction time range is from 0.5 to 6 hours.
